(19) United States
(12) Patent Application Publication
(10) Pub. No.: US 2010/0317963 A1
(43) Pub. Date: Dec. 16, 2010

(54) ENDOSCOPIC ULTRASOUND-GUIDED STENT PLACEMENT DEVICE AND METHOD

(76) Inventor: Michael S. Clancy, Monaleen (IE)

Correspondence Address: BRINKS HOFER GILSON & LIONE/CHICAGO/COOK, PO BOX 10395, CHICAGO, IL, 60610, US

(21) Appl. No.: 12/794,985

(22) Filed: Jun. 7, 2010

Related U.S. Application Data
(60) Provisional application No. 61/186,593, filed on Jun. 12, 2009.

(51) Int. Cl. A61B 8/12; A61F 2/84

(52) U.S. Cl. 600/424; 623/1.11

(57) ABSTRACT

A system and method for ultrasound-guided placement of a stent are provided. The system includes an echogenic cannula element that may include an echogenically-enhanced cannula that may be embodied as a piercing-tipped needle and/or an echogenically-enhanced stylet, where an echogenically-enhanced cannula element portion is near the distal cannula end. A stent is disposed about the echogenically-enhanced cannula element portion, such that the stent can be navigated to a target site using ultrasound imaging of the echogenically-enhanced cannula element portion.

### CROSS-REFERENCE TO RELATED APPLICATIONS

This application claims priority to U.S. Provisional Application Ser. No. 61/186,593, filed Jun. 12, 2009, which is incorporated herein by reference in its entirety.

### TECHNICAL FIELD

The invention relates generally to methods of minimally-invasive surgical methods. More particularly, the invention pertains to a system and method for ultrasound-guided placement of a stent.

### BACKGROUND

The development of minimally invasive methods and devices over recent years has revolutionized the practice of medicine. These methods and devices allow clinicians to perform a wide variety of procedures while minimizing trauma to the patient. Along these lines, there is a need for devices and methods that employ minimally invasive technologies in order to access occluded regions in a mammalian body that may not be visible even from a minimally invasive device such as an endoscope. This is a particular challenge when a target region needs to be identified with sufficient locational specificity that visualization is preferable, but video visualization (e.g., via a camera or other video element of an endoscope) may be impractical. It is generally desirable to minimize the patient and caregiver exposure to radiation during use of fluoroscopic visualization—particularly for an extended time period, such as may be required to navigate a stent to a target site in the patient's biliary tree.

Stent devices are known and used for maintaining the patency of the biliary tree, or common bile duct, and for treatment of other ailments related to the biliary tree (including the gall bladder and pancreas). FIG. 1 is a partial diagrammatic view (in longitudinal/coronal section) of a biliary system 100 showing the common bile duct 102, the gall bladder 108, the common hepatic duct 103, the duodenum 110 and the pancreas 112.

Strictures or occlusions that develop in the upper common bile duct, the common hepatic duct, and/or the left and right hepatic ducts can interfere with the proper drainage of those ducts. FIG. 2 depicts the same view of the biliary system 100 as FIG. 1, but showing a stricture 114 within the common hepatic duct 103. A successful method of treatment to reestablish proper drainage through the diseased ducts has been to open the ducts by placing prostheses, such as self-expanding or other biliary stents (e.g., polymer stents with or without one or two pigtails), within the restrictions. Other ailments associated with the biliary tree can be treated by placement of a stent. One example is pancreatic pseudocysts, which may form in the pancreas as a pocket holding necrotic tissue, blood, and pancreatic secretions. They may occur, for example, in connection with pancreatitis or as the result of abdominal injury, and are differentiated from true cysts by being contained by a fibrous and/or granular tissue capsule rather than an epithelial lining.

FIG. 3 illustrates a mesh-type stent 116 in the common hepatic duct 103. Such stents 116 may be metallic and/or include one or more fiducials used for fluoroscopic visualization during installation (as visualization via the camera of an endoscope is not possible after the stent is directed through the sphincter of Oddi 118 into the common bile duct 102). However, these and polymer stents often do not provide sufficient difference in ultrasound reflectivity (relative to the surrounding tissues) to accurately direct and orient a stent under ultrasound visualization. As a result, some prior art stents and similar access devices are difficult to navigate to a precise location within the body (the biliary tree being one example for illustrative purposes only). This difficulty often occurs due to the lack of a guidance system configured to indicate where the device is located without introducing the risks associated with over-use of fluoroscopy.

In addition, the procedure described above—similar to other procedures for accessing the gall bladder, a pancreatic pseudocyst, or other structure in need of access for diagnostic and/or therapeutic purposes—presently requires multiple steps and tools. Typically, one or more cannulation and/or dilator devices must be introduced to open the stricture, penetrate the pseudocyst, or otherwise provide a sufficient path of access for stent-introduction. As with most steps/method described here, access to the duodenum adjacent the biliary tree is commonly provided through a working channel of an endoscope such as, for example, a side-viewing duodenoscope. For a variety of reasons, including the visualization challenges noted above, after the cannulator and/or dilator devices are withdrawn, it may be difficult to accurately navigate and position a stent in an optimal orientation. If the procedure includes treatment of a cyst (for example, placement of a double-pigtail stent to treat a pseudocyst in the pancreas), a needle or cystotome may have to have been introduced and withdrawn (in which case, drainage/decompression of the cyst may further hamper introduction of a wire guide and stent in the manner typically used for stent placement).

It would be beneficial to provide minimally access devices and methods that will alleviate one or more of these or other problems.

### BRIEF SUMMARY

A device and method are provided including system and method for ultrasound-guided placement of a generally non-echogenic stent. The system preferably includes an echogenic cannula element that may include an echogenically-enhanced cannula and/or an echogenically-enhanced stylet, where an echogenically-enhanced cannula element portion is near the distal cannula end. A stent may be disposed around the echogenically-enhanced cannula element portion, such that the stent can be navigated to a target site using ultrasound imaging of the echogenically-enhanced cannula element portion.

### BRIEF DESCRIPTION OF THE DRAWINGS

The invention may be better understood with reference to the following drawings and description. The components in the figures are not necessarily drawn to scale, emphasis instead being placed upon illustrating the principles of the invention.

FIG. 1 shows a partial diagrammatic view (in longitudinal/coronal section) of a biliary system;

FIG. 2 shows the biliary system of FIG. 1 with a duct-constricting disease condition;

FIG. 3 shows the biliary system of FIG. 1 with a duct-constricting disease condition, treated by a stent;

FIG. 4 shows an echogenic needle assembly embodiment including a magnified view of a distal portion having a beveled needle for use with the present method;

FIG. 5 shows a detail view of the beveled echogenic needle of FIG. 4;

FIG. 6 shows a detail view of a round-tipped cannula device for use with the present method;

FIG. 7 shows a section view of an echogenic-stylet cannula device for use with the present method;

FIG. 8 shows an assembled cannula device with stent, configured for use with the present method;

FIGS. 8A and 8B show, respectively, a single-pigtail stent and a double-pigtail stent, either of which may be used with the present method (as may straight or slightly-curved stents);

FIGS. 9A-9B show steps of the present method; and

FIG. 10 shows an echogenic cannula assembly embodiment including a magnified view of a distal portion having a beveled cannula for use with the present method.

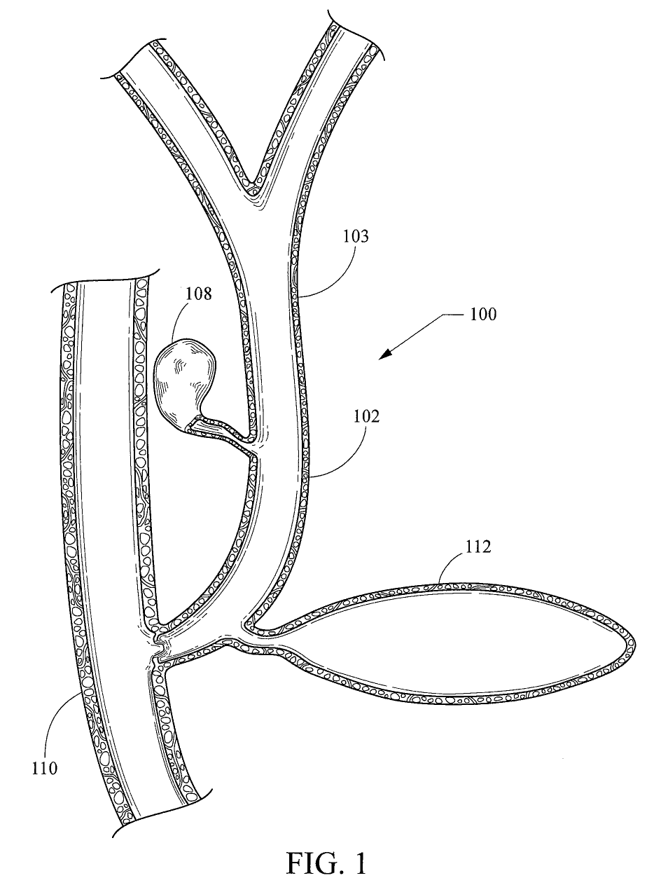

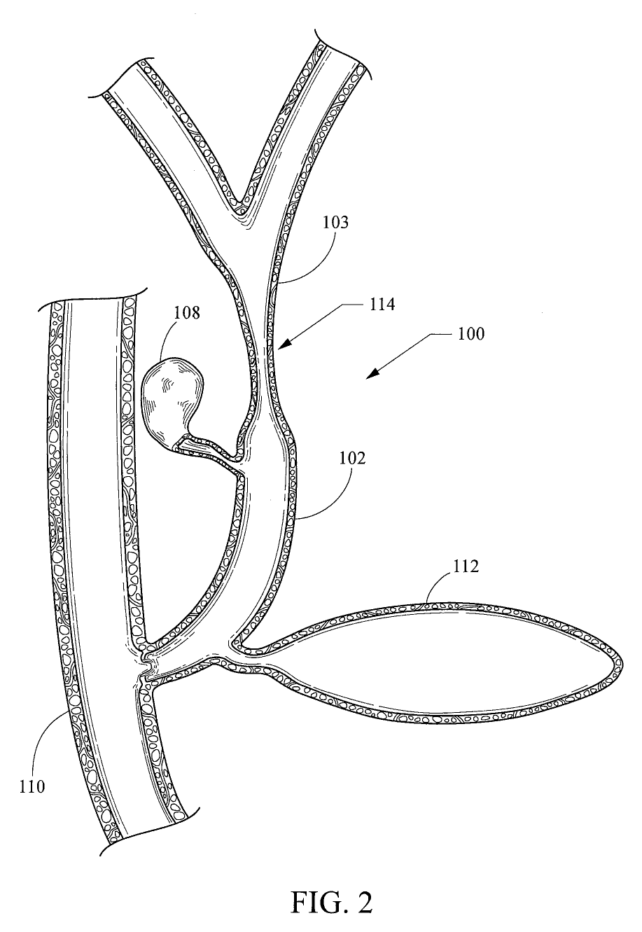

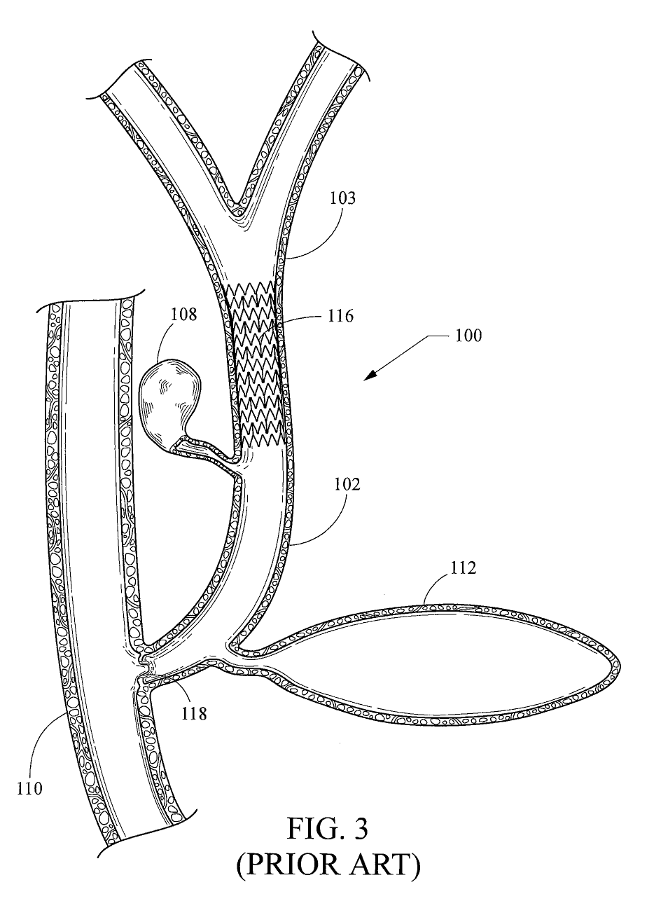

### DETAILED DESCRIPTION

As used herein, including in the claims, the term “echogenic” is defined as having enhanced echogenicity. Specifically, it is used to refer to materials or portions of materials that are constructed or are treated to have greater reflectivity of ultrasonic waves than standard materials used for a stent, sheath, cannula, catheter, and/or stylet, and to provide an echogenic profile relative to surrounding tissues during use in a patient body to accurately orient and direct the echogenic device portion. It is known in the art that most materials used for a stent sheath, catheter, cannula, or stylet will reflect some ultrasonic waves, but the term “echogenicity,” as used herein includes treating the surface by creating a textured or patterned surface including, for example, one or more of dimples, divots, knurling, ridges, or the like—each of which is known in the art to enhance echogenicity as compared to a smooth surface for a similarly-sized/shaped object, (and/or, when specifically referenced, using a material known to provide an enhanced echogenic profile) configured to provide clear ultrasound visualization at a resolution providing for accurate location and navigation of a device in a body (e.g., of a patient). Echogenic construction may be enhanced by surface texture, but can also be provided by structural inclusions such as embedded bubbles, beads, or other inclusions in a polymer or metal that can provide for a different ultrasound reflectivity than material surrounding them. Also, as used herein, the term “needle” refers generally to a tubular cannula that may or may not have a piercing distal tip, and the term “cannula” may refer to a rigid or flexible tubular device that may include a piercing tip. As used throughout unless a special exception is specifically identified, the term “proximal” refers to an end or direction nearer a physician or other person handling an object during normal use (generally, the “handle end”), and “distal” refers to the opposite end (generally, the “tool end”). Drawing figures are not necessarily to scale, as various parts thereof may be magnified or otherwise emphasized to clarify structural features (including that, as one specific example, the proportions of the duodenum and biliary tree structure are not shown to scale, as those of skill in the art will know that the biliary tree is proportionally smaller than shown).

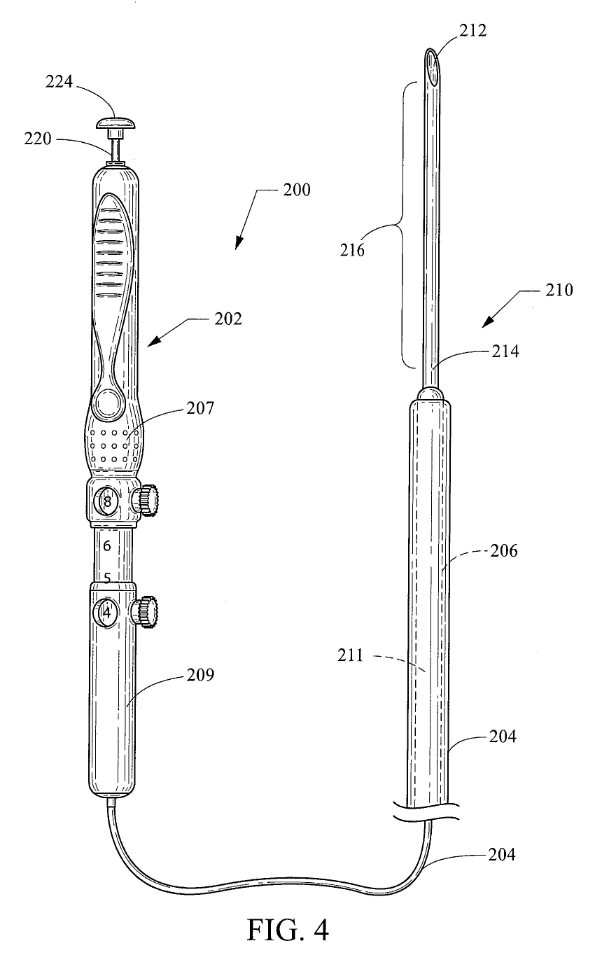

An embodiment of a method for echogenic placement of a stent is described with reference to FIGS. 4-9B. It should be appreciated that implantable devices other than stents may be used within the scope of the present method. The method may be considered useful in placement of a generally non-echogenic implantable device (i.e., one that does not provide a consistently resolvable image under ultrasound visualization, such as would be most useful for navigating it and orienting it during placement).

The ease and efficiency of the present novel method should be appreciated in contrast with the existing methods described in the background. Reducing the number of tools required and the amount of time needed for placement of a stent provides numerous advantages over existing methods. Those advantages include that the patient is subjected to the discomfort and inconvenience (anesthesia, etc.) for a lesser time, the cost of materials consumed will be reduced for the patient, and the amount of time required by a treating physician and attending staff will be reduced, providing cost and efficiency benefits (that may also reduce patient and hospital costs associated with a stent-placement procedure).

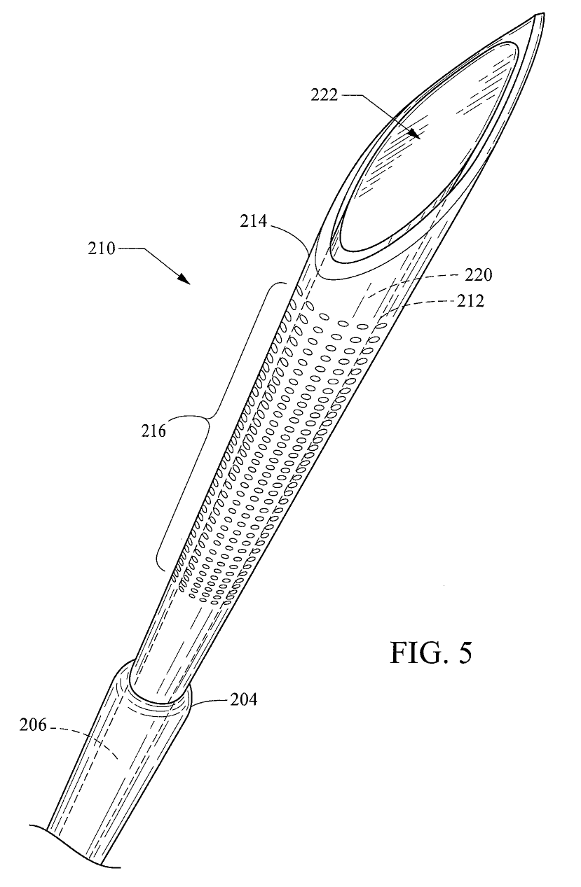

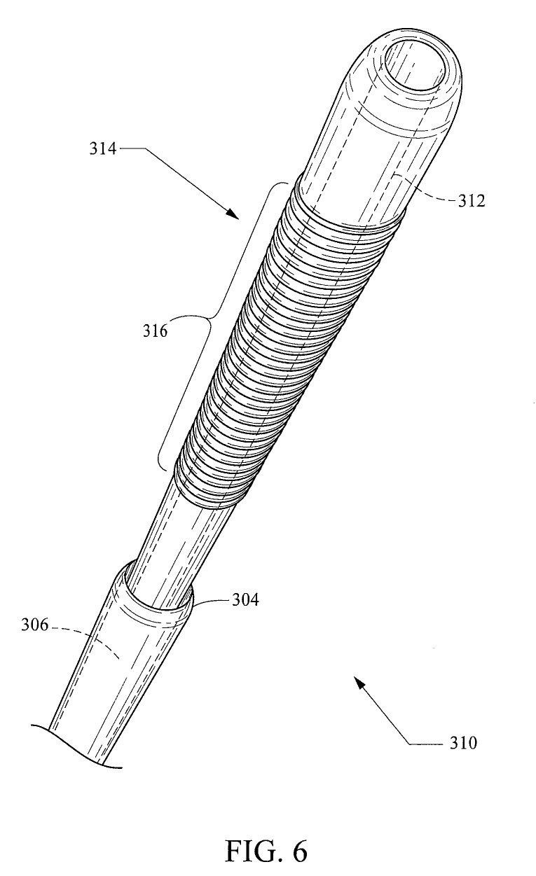

FIGS. 4-6 show examples of echogenic needle assemblies 210, 310 useful in practicing the method. In FIG. 4, the assembly is shown with a handle, and the distal portion is somewhat enlarged for clarity of detail, and in FIGS. 5-6, different specific needle embodiments are shown in greater detail. One example of a needle assembly useful for the present method is an EchoTip® needle (available from Cook Medical, Bloomington, Ind.) in its commercially available form, or adapted as desired for a particular user. The primary difference between the embodiments as illustrated herein is the distal needle tip configuration (beveled versus rounded), but it will be appreciated that other commercially available products may be used within the scope of the presently claimed method, and the particular needle assemblies 210, 310 set forth here are provided only as illustrative examples.

FIG. 4 shows a beveled-needle configuration of an echogenic needle assembly 200. The needle assembly 200 includes a proximal handle 202, an elongate outer needle sheath 204, an elongate hollow needle structure 210 extending through a sheath lumen 206 of the needle sheath 204, and an elongate stylet 220 extending through a needle lumen 212 of the needle structure 210. As shown in greater detail in FIG. 5, the distal portion of the needle structure 210 includes a beveled needle 214 that is constructed of metal and that includes a textured irregular surface region 216 along its cannula shaft illustrated here as being dimpled. The dimpled region 216 is configured to enhance the echogenicity of the needle 214. In other embodiments, the needle 214 may have ridges or other surface details on an internal and/or external surface to provide enhanced echogenicity, and may be constructed of an appropriate material other than metal. The needle structure 210 may include a flexible body 211 extending proximally from the needle 214 through the sheath lumen 206. Needles for use with the present method may be constructed of a polymer or metal material, and preferred embodiments may be constructed of a nickel-titanium alloy and/or stainless steel.

Those of skill in the art will appreciate that numerous structures and techniques are known that will provide enhanced echogenicity for a needle, any of which may be used in a needle assembly within the scope of the present invention. Examples of structures adaptable for echogenic needles (including needle/stylet systems) that may be useful within the scope of the present method could include, for example, those disclosed in U.S. Pat. App. Pub. Nos. 2003/0073902; 2003/0158480; 2008/0097213; U.S. Pat. Nos. 4,582,061; 6,018,676; 6,610,016; and PCT Pat. App. Pub. Nos. WO96/027329; WO07/067324; and WO08/062451, each of which is incorporated by reference herein.

The distal tip 222 of the stylet 220 preferably aligns with the beveled end of the needle 214 such that the needle has enhanced structural support and can penetrate tissue and/or cannulate an occluded lumen without coring tissue that is being cannulated. The handle 202 includes a proximal stylet-removal portion 224, which will allow a user to remove the stylet 220 at a desired time. Upon removal of the stylet 220, a user may introduce material (e.g., radio-opaque contrast fluid, a wire guide, a different-length stylet) or withdraw material through the needle lumen 212.

The handle 202 also includes a needle-attached first handle portion 207 that is attached to the needle structure 210 and all of which is slidable relative to a sheath-attached second handle portion 209. This structural configuration allows the needle structure 210 to be drawn proximally through the sheath lumen 206 and/or the needle sheath 204 to be advanced distally along the needle structure 210. The embodiment illustrated in FIG. 4 includes an indexing register on the handle 202, whereby a user may accurately measure in centimeters the relative movement between the needle sheath 204 and the needle structure 210.

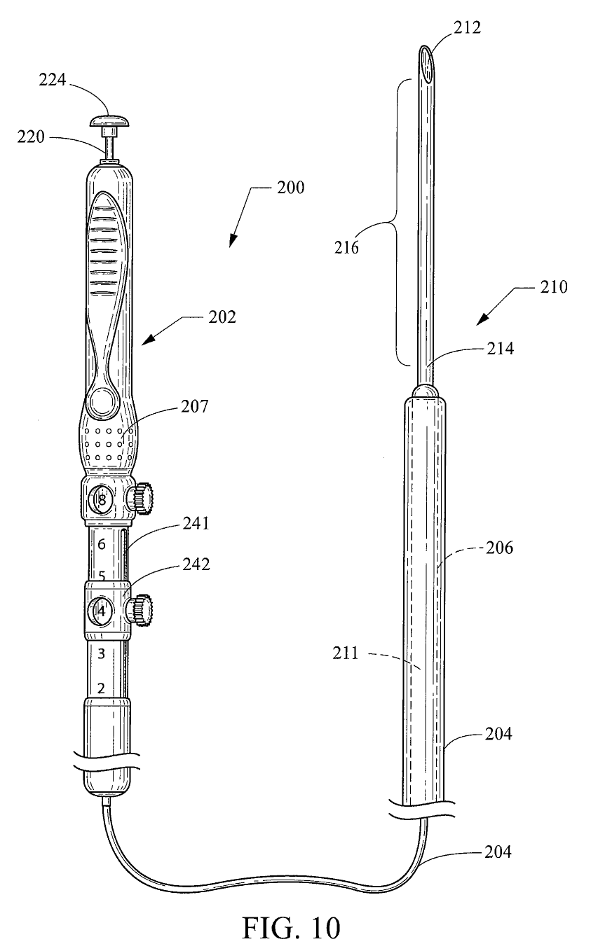

FIG. 10 shows another embodiment of the handle 202 of FIG. 4. In the embodiment of FIG. 10, the handle 202 includes a sheath-manipulation slot 241 through which a sheath-manipulation ring 242 is attached to the sheath 204 and slides up and down along the handle body in a manner that moves the sheath 204 longitudinally relative to the handle 202 and the needle structure 210. With this configuration, the sheath 204 and needle structure 210 can be more easily manipulated lengthwise relative to each other.

FIG. 6 shows a detail view of a round-tipped needle configuration of an echogenic needle assembly that may be—but for the round-tipped needle—otherwise similar to the assembly 200. The needle structure 310 includes extends distally out of an elongate outer sheath 304. The elongate hollow needle structure 310 includes a long flexible body (not shown) extending through a sheath lumen 306 of the sheath 304, and may include an elongate stylet extending through a needle lumen 312 of the needle structure 310. The distal portion of the needle structure 310 includes a rounded needle 314 that includes an irregular surface region 316 illustrated here as being ribbed. The ribbed region 316 is configured to enhance the echogenicity of the needle 314. In other embodiments, the needle 314 may have dimples, edged ridges, knurling, and/or regular or irregular other surface texturing or details on an internal and/or external surface to provide enhanced echogenicity, and may be constructed of any appropriate material. The rounded tip may be preferred when loading a stent onto the needle because—particularly for curved stents (up to and including pigtail stents)—the sharpened point of a beveled needle may damage the stent and/or impair its ability to easily be placed onto the needle. A rounded tip may also prevent stripping of a wire guide passing though the needle lumen. Those of skill in the art will appreciate that numerous structures and techniques are known that will provide enhanced echogenicity for a needle, any of which may be used in a needle assembly within the scope of the present invention.

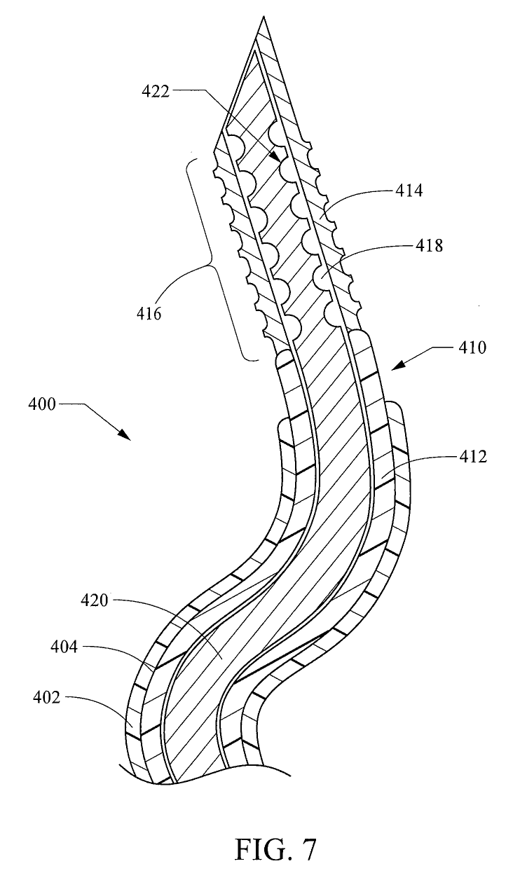

FIG. 7 shows a distal-end detail longitudinal section view of a stent-placement system 400 with an outer sheath 402 (handle end not shown). The overall structure of the system may generally be similar to the needle assembly 200, or adapted from other devices appropriate for use with the present method. The outer sheath 402 includes a sheath lumen 404. An echogenic needle assembly 410 extends through the sheath lumen 404. The echogenic needle assembly 410 includes a flexible body length 412 with bevel-tipped distal needle 414 attached to and extending distally from it. The distal needle 414 includes a textured surface 416 with a needle lumen 418. The textured surface may alternatively be knurled, ridged, ribbed, pitted, or otherwise textured in a manner configured to enhance reflection of ultrasound waves, thereby providing echogenicity that preferably is greater than a non-textured surface of the same material (e.g., metal alloy). Preferably, it is disposed immediately adjacent the distal needle end to provide for accurate echogenic visualization and navigation of the needle. In other embodiments, the needle 414 may be coated with an echogenic polymer such as is disclosed in U.S. Pat. No. 6,610,016, which is incorporated herein by reference. Metal alloys, various polymers, and other materials generally include inherent echogenicity, but it is preferable that such echogenicity be enhanced in one or more ways set forth herein or yet to be developed.

Although the textured surface is illustrated as being the outer surface, in addition (or alternatively), a textured surface, polymer coating, or other echogenicity-enhancing means may be disposed on the outer surface, the inner surface (e.g., the surface defining the needle lumen 418), and/or included within the wall defining the needle 414. A stylet 420 extends through the needle lumen 418. The stylet 420 is also shown as including an echogenicity-enhancing surface (preferably co-extensive with an echogenic region, e.g. 416, of the overlying needle—if present—when the stylet is fully inserted therein) that is patterned with a series of circumferential scallops 422 that may alternatively be embodied as another texture, shape, pattern, or a coating material that would enhance echogenicity of the stylet 420 whether or not it is used with an echogenic needle.

In other embodiments, one or both of the needle 410 and stylet 420 may have echogenic inclusions in their solid portions such as, for example small pockets of air or vacuum space that would create an “internal texture” configured to enhance echogenicity of that device by providing ultrasound-reflective surfaces within and/or upon its structure. As used herein, the phrase “echogenic needle portion” means the portion of the needle assembly provided with one or more echogenicity-enhancing means, whether disposed in or on a stylet or needle. In any event, the echogenic needle portion preferably is configured to provide reflection of ultrasonic waves sufficient for ultrasonic imaging of the echogenic needle portion at a resolution providing for effective navigation of an overlying stent disposed thereon in a patient body. The outer sheath may also include an echogenic material (including an echogenic surface pattern and/or construction).

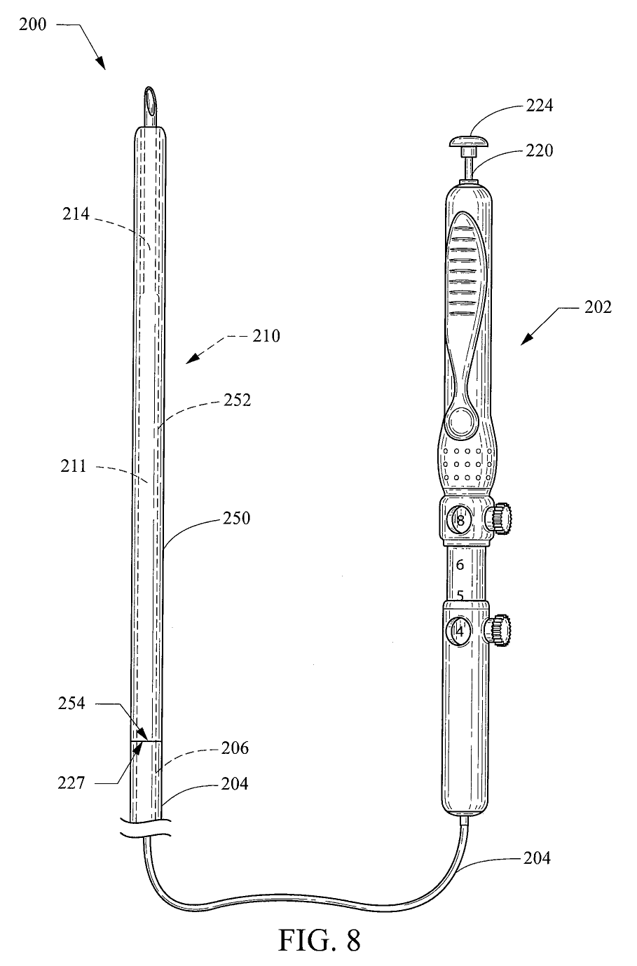

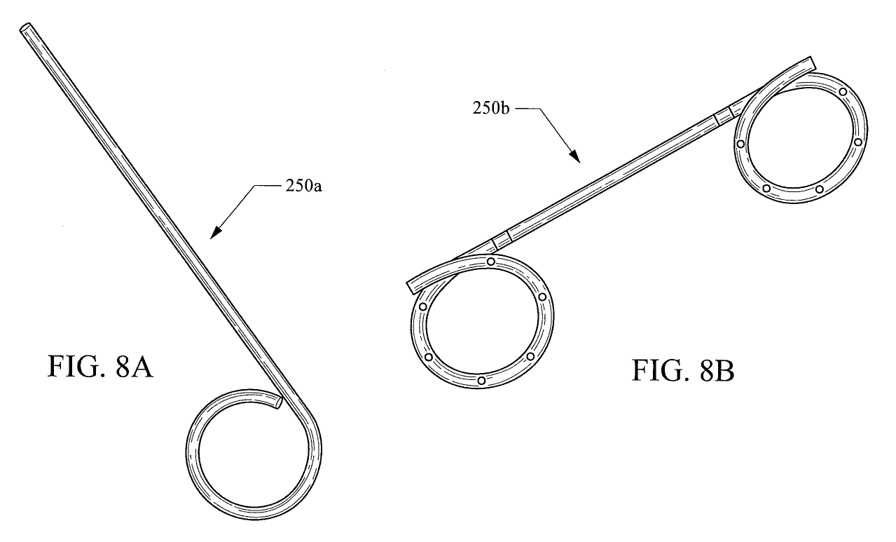

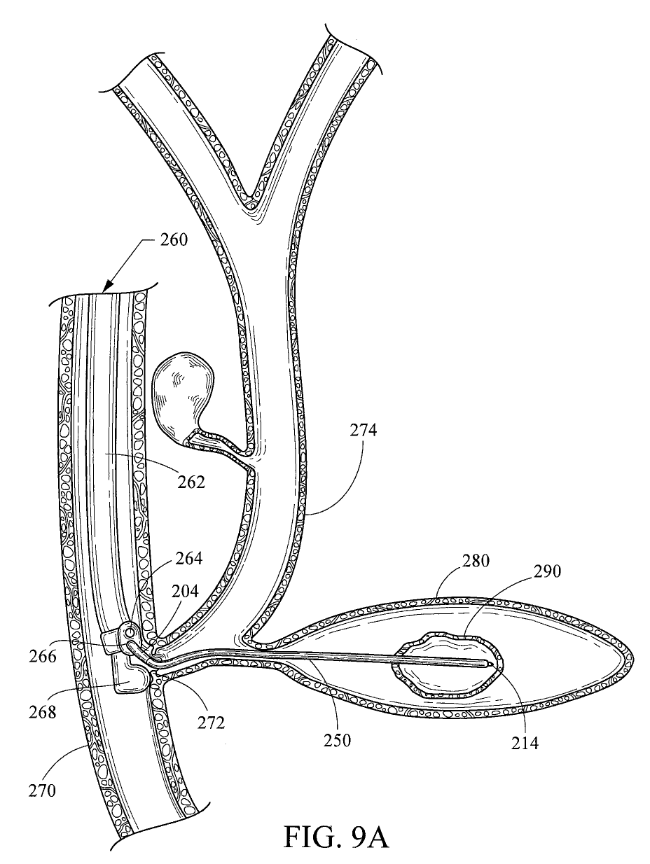

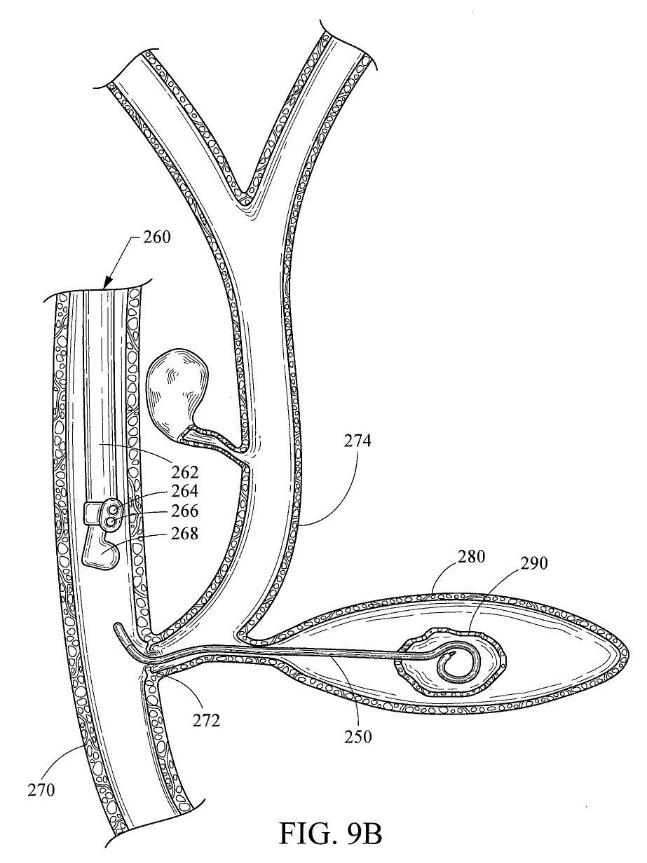

FIG. 8 shows a system for using the presently-described method, using the same needle assembly 200 as shown in FIG. 4, with a stent 250 placed upon a distal region including most of the length of the needle 214 (except for, preferably, the distal tip which should be left uncovered for use in piercing and cannulating tissue for placing the stent). As in FIG. 4, the distal portion is shown enlarged for clarity (and, as in the other figures, neither the enlarged nor the non-enlarged portion of the drawing is necessarily to scale). The stent 250 is shown as polymer pigtail stent in this drawing and in FIGS. 9A-9B, but it should be appreciated that other polymer stents, or stents constructed of other materials—whether curled, curved, or straight—may be used within the scope of the presently-claimed invention. The stent may be a single-pigtail stent 250a as shown in FIG. 8A, or a double-pigtail stent 250b as shown in FIG. 8B. The stent 250 includes a stent lumen 252 through which the needle structure 210 extends in a manner preferably maintaining a friction grip that prevents the stent 250 from falling off, but that will allow the stent to be advanced distally relative to the needle structure 210 by advancement of the distal end 227 of the needle sheath 204 against its proximal stent end 254 and/or retraction of the needle structure 210 relative to the needle sheath 204 and stent 250.

A method for endoscopic, ultrasound-guided delivery of a stent device to a target site is described with reference to FIGS. 4, 5, 8, and 9A-9B. The target site preferably is a location in or near the gastrointestinal tract (e.g., liver, pancreas) such as those locations that may be visualizable by endoscopy (using a minimally invasive endoscope introduced through a natural patient orifice, e.g., mouth, anus, vagina), and may be most useful in those locations that are more readily visualizable by ultrasound endoscopy where the video element of an endoscope may not provide ideal visualization. This includes—more broadly—sites reachable through NOTES (natural orifice translumenal endoscopic surgery) procedures. The present method may also be used with other minimally-invasive surgical techniques such as percutaneous endoscopic procedures (e.g., laparoscopic procedures), but most preferably is used with the less invasive endoscopy procedures.

The present method presents advantages of using ultrasound for visualization of the procedure, which is typically more desirable than using fluoroscopy alone. In part, using ultrasound can reduce—or in some cases eliminate—the need during a procedure for the patient and treating personnel (physician, assisting personnel, etc.) to be exposed to the radiation of a fluoroscopic visualization procedure. This can be even more desirable for patients who may be undergoing radiation therapy to treat a disease condition and for whom it is even more desirable to limit procedural radiation exposure to keep their overall exposure minimized. Similarly, it is preferable to minimize the radiation exposure of treatment personnel.

The method is described particularly with reference to the ultrasound-visualizable endoscopic stent-placement system 200 of FIGS. 4, 5, and 8. As shown in FIG. 9A, an endoscopic ultrasound (EUS) endoscope 260 is provided including a scope body 262, a camera (e.g., CCD or other video unit for direct visualization) 264, a working channel 266, and an ultrasound probe 268. The flexible outer needle sheath 204 includes a longitudinal sheath lumen 206 extending from a distal sheath end 227 toward a proximal end (not shown) of the sheath. A needle assembly 210 extends through the sheath lumen 206.

The needle assembly 210 includes a distal needle 214 configured for longitudinal extension from the distal sheath end 227 and including a longitudinal needle lumen 212 therethrough. A flexible body length 211 extends proximally from the distal needle 214 through the sheath lumen 206. A stylet 220 extends through the body length 211 and the needle lumen 212. The echogenic needle portion 216 is disposed immediately adjacent the distal needle end. The echogenic needle portion 216 includes a dimpled surface configured to enhance reflection of ultrasound for visual resolution of the needle 214.

The stent device 250 is disposed coaxially around the echogenic needle portion 216 such that the echogenic needle portion extends through at least a portion of a longitudinal stent lumen 252. The echogenic needle portion 216 is configured to provide reflection of ultrasonic waves sufficient for ultrasonic imaging of the echogenic needle portion 216 at a resolution providing for effective navigation of the stent 250 disposed thereon in a patient body.

Ultrasound sonography preferably is used to provide visualization of the echogenic needle portion and a target site. This may be done by one or both of an endoscope-mounted ultrasound transducer (e.g., the ultrasound probe 268) and an external (to the patient body) ultrasound transducer. The system preferably is directed as far as possible under direct visualization (through the video element 264 provided in the scope 260, supplemented or replaced as desired by the physician and assisting personnel). When the system is directed to an area that cannot be visualized under endoscopic video visualization (e.g., into the biliary tree), it is navigated under ultrasound visualization (which may also be used in conjunction with the video visualization). Under ultrasound navigation (which may be supplemented by fluoroscopic visualization if needed and if the radiation exposure to patient and treating personnel is justified) a user directs the echogenic needle portion 216 and the overlying stent 250 to a location near a target site 290 such as, for example, a pancreatic pseudocyst 290 in a patient pancreas 280. It should be appreciated that the echogenic needle portion 216 may, in a different embodiment (e.g., like FIG. 7) include or be provided by an echogenic stylet portion. In any embodiment, it preferably provides an ultrasound signature that allows accurate location and navigation of the stent 250 disposed over the echogenic region 216 of the needle 214. This remains true for an embodiment of the method not using an endoscope such as, for example, placing a stent into the neck of a patient's urinary bladder, where the ultrasound visualization is not conducted endoscopically.

Then, using the ultrasound visualization, the user may then direct the distal needle and the overlying stent to a location nearer the target site where it is desired to place the stent 250. In FIG. 9A, the endoscope 260 has been directed though a patient's duodenum 270 to a site adjacent the target site. A distal portion of the stent-encompassed needle assembly 210 has been directed through the sphincter of Oddi 272 and a portion of the common biliary duct 274, then into the pseudocyst 290 in the pancreas 280. Next, the user can move at least one of the distal sheath end 227 and distal needle 214 relative to the other to place the stent 250 into a desired location. That is, the needle 214 may be advanced (with the stent 250 disposed thereupon) into the desired location, then the needle 240 can be withdrawn from the stent lumen 252 while the distal end 227 of the needle sheath 204 is held in position to prevent retraction/withdrawal of the stent 250, thereby deploying it. This method is generally preferred for accurate placement of the stent. FIG. 9B shows the stent 250 as having been deployed, and the needle assembly 200 withdrawn into the working channel 266 of the endoscope 260. Alternatively, the needle sheath 204 may be advanced over the needle 214, pushing the stent 250 off from it (thereby functioning as a pusher in a manner well-known in the stent-placement art). After the stent 250 is placed, the entire apparatus 200 may be withdrawn.

Those of skill in the art will appreciate that embodiments not expressly illustrated herein may be practiced within the scope of the present invention, including that features described herein for different embodiments may be combined with each other and/or with currently-known or future-developed technologies while remaining within the scope of the claims presented here. It is therefore intended that the foregoing detailed description be regarded as illustrative rather than limiting. And, it should be understood that the following claims, including all equivalents, are intended to define the spirit and scope of this invention.

We claim:

1. An ultrasound-visualizable endoscopic stent-placement system, comprising:
a flexible outer sheath including a longitudinal sheath lumen extending from a distal end toward a proximal end of the sheath; and
a cannula assembly extending through the sheath lumen, the cannula assembly including
a distal cannula configured for longitudinal extension from a distal sheath end and including a longitudinal cannula lumen therethrough;
a flexible body length extending proximally from the distal cannula through the sheath lumen;
a stylet extending through the body length and the cannula lumen; and
an echogenic cannula portion disposed immediately adjacent a distal cannula end; and
a stent device disposed coaxially around the echogenic cannula portion such that the echogenic cannula portion extends through at least a portion of a longitudinal stent lumen of the stent device, wherein the echogenic cannula portion is configured to provide reflection of ultrasonic waves sufficient for ultrasonic imaging of the echogenic cannula portion at a resolution providing for effective navigation of the stent disposed thereon in a patient body.

2. The system of claim 1, wherein the echogenic cannula portion comprises an ultrasound-reflective material comprised by at least one of an inner cannula surface, an outer cannula surface, a cannula body, a stylet surface, a stylet body, a stylet coating, and a cannula coating.

3. The system of claim 2, where an ultrasound-reflective material is disposed on the outer surface of the echogenic cannula portion.

4. The system of claim 2, where the ultrasound-reflective material is selected from the group consisting of: a pattern-surfaced material; an echogenic polymer; and any combination thereof.

5. The system of claim 1, where the echogenic cannula portion comprises a textured surface of at least one of the distal cannula and the stylet.

6. The system of claim 1, where the outer sheath comprises an echogenic material.

7. The system of claim 6, where the echogenic material is selected from an echogenic polymer and an alloy metal, the alloy metal comprising at least one textured echogenic surface.

8. A method for endoscopic, ultrasound-guided delivery of a stent device to a target site, the method comprising the steps of:
providing the system of claim 1;
providing ultrasound visualization of the echogenic cannula portion and the target site;
directing the echogenic cannula portion and the overlying stent to a location near a target site;
directing the distal cannula to a location nearer the target site where it is desired to place the stent device; and
moving at least one of the distal sheath end and the body length relative to the other of the distal sheath end and the body length to deploy the stent device into a desired location.

9. The method of claim 8, wherein the steps of directing the distal cannula and moving at least one of the distal sheath end and the body length are executed substantially simultaneously.

10. The method of claim 8, further comprising a step of withdrawing proximally the distal cannula and flexible body length from the stent lumen to deploy the stent device.

11. The method of claim 10, wherein the outer sheath is held substantially in place relative to the stent device as the distal cannula and flexible body are proximally withdrawn such that it functions as a holder/pusher to aid deployment of the stent device.

12. The method of claim 8, further comprising a step of withdrawing the stylet from the cannula lumen.

13. The method of claim 12, further comprising a step of introducing a contrast fluid through the cannula lumen.

14. The method of claim 12, further comprising a step wherein a wire guide is directed to the target site, and the system is directed over the wire guide, where the cannula lumen is used as a wire guide lumen.

15. The method of claim 8, wherein the target site is located in a pancreas, liver, or biliary tree duct of a patient.

16. The method of claim 8, where the stent device comprises a single-pigtail stent.

17. The method of claim 8, where the stent device comprises a double-pigtail stent.

18. The method of claim 8, wherein a distal tip of the distal cannula comprises a beveled tissue-penetrating point.

19. The method of claim 8, wherein a distal tip of the distal cannula comprises a rounded cannulating point, the rounding configured to minimize at least one of a potential cutting of tissue, damage to a wire guide passing through the cannula lumen, and damage to the stent device during passage of the stent device over the distal tip of the distal cannula.

20. A method for ultrasound-guided delivery of a stent device to a target site, the method comprising the steps of:
providing an ultrasound-visualizable stent-placement system, the system comprising:
a flexible outer sheath including a longitudinal sheath lumen extending from a distal end toward a proximal end of the sheath; and
a cannula assembly extending through the sheath lumen, the cannula assembly including
a distal cannula configured for longitudinal extension from the distal sheath end and including a longitudinal cannula lumen therethrough;
a flexible body length extending proximally from the distal cannula through the sheath lumen;
a stylet extending through the body length and the cannula lumen; and
an echogenic cannula portion disposed immediately adjacent a distal cannula end, the echogenic cannula portion comprising a dimpled surface configured to enhance reflection of ultrasound for visual resolution of the cannula; and
a stent device disposed coaxially adjacent the echogenic cannula portion such that the echogenic cannula portion extends through at least a portion of a longitudinal stent lumen of the stent device, wherein the echogenic cannula portion is configured to provide reflection of ultrasonic waves sufficient for ultrasonic imaging of the echogenic cannula portion at a resolution providing for effective navigation of the stent disposed thereon in a patient body;
providing ultrasound visualization of the echogenic cannula portion and a target site;
directing the echogenic cannula portion and the overlying stent to a location near a target site;
using the ultrasound visualization to direct the distal cannula and the overlying stent to a location nearer the target site where it is desired to place the stent device; and
moving at least one of the distal sheath end and distal cannula relative to the other to place the stent device into a desired location.
